(19) United States
(12) Patent Application Publication
(10) Pub. No.: US 2010/0063156 A1
(43) Pub. Date: Mar. 11, 2010

(54) ANANDAMIDE

(76) Inventor: Hugo Streekstra, Amsterdam (NL)

Correspondence Address: NIXON & VANDERHYE, PC, 901 NORTH GLEBE ROAD, 11TH FLOOR, ARLINGTON, VA, 22203, US

(21) Appl. No.: 12/443,721

(22) PCT Filed: Oct. 3, 2007
(86) PCT No.: PCT/EP07/60499

§ 371 Date: Mar. 31, 2009

(30) Foreign Application Priority Data
Oct. 5, 2006 (EP) 06121796.4

(51) Int. Cl. A61K 31/16; C07C 233/20; A61P 3/00

(52) U.S. Cl. 514/627; 564/208

(57) ABSTRACT

The present invention describes provides the use of anandamide for the manufacture of a nutraceutical for oral intake preferably a medicament for reducing appetite, giving a satiety effect, preventing or reducing inflammatory bowel disease or preventing or reducing irritable bowel syndrome

### BACKGROUND OF THE INVENTION

Anandamide is an endogenous compound in the body of humans and animals. It is the natural ligand of the cannabis receptors CB1 and CB2 [Pertwee (2001) Cannabinoids and the Gastrointestinal Tract; Gut 48:859-867]. It is a representative of the fatty acid ethanolamides, a group of molecules that is increasingly being associated with the regulation of various physiological and neurological functions. Anandamide is not the only endogenous ligand of the CB receptors: 2-arachidonyl-glycerol (2-AG) is believed to be an even more potent ligand. 2-AG represents another class of physiologically active compounds, the 2-mono-acyl-glycerols. Anandamide is formed in the body by N-acylation of the phospholipid phosphatidyl-ethanolamine with arachidonic acid, followed by hydrolysis of the phosphatidyl-group [Okamoto et al. (2004) Molecular Characterization of a Phospholipase D Generating Anandamide and Its Congeners; J. Biol. Chem. 279:5298-5305]. Once formed, anandamide can be hydrolyzed by a dedicated amidase, the fatty acid amide hydrolyase. In contrast to the plant-derived cannabinoids (e.g. marijuana) and some synthetic cannabinoids, anandamide is constantly being synthesized and degraded in the human body. Therefore, its activity at the receptor is regulated, and attenuated compared to the strong effects exerted by the more stable alien ligands. Stimulation of the CB receptors in the brain is believed to have a mood-enhancing effect and to increase appetite. It has been suggested that anandamide could be useful when lack of appetite is a problem, such as in old or sick people [Williams & Kirkham (2002) Observational Analysis of Feeding Induced by Δ⁹-THC and Anandamide; Physiol. Behay. 76:241-250]. Appetite stimulating effects were found—in rats—when anandamide was introduced by injection. In another study, it was found that injected anandamide could promote extra feeding in animals, in this case partially satiated rats [Gomez et al. (2002) A Peripheral Mechanism for CB1 Cannabinoid Receptor-Dependent Modulation of Feeding; J. Neurosci 22:9612-9617].

In general, it is believed that anandamide produces only weak and transient cannibinoid effects in vivo, probably as a result of its rapid catabolism, limiting its effectiveness as a means of treatment [Harrold & Williams (2003) The Cannabinoid System: a Role in Both the Homeostatic and Hedonic Control of Eating?; Br. J. Nutr. 90:729].

Although stimulation of appetite may be very useful under certain circumstances, preparations that would have the opposite effect—reduce appetite and/or the amount of food consumed and/or the accumulation of fat in the body—are considered to be particularly interesting nowadays, taking into account the trend towards increased obesity in populations all over the world.

In U.S. Pat. No. 6,911,474, it is described that some fatty acid ethanolamides reduced voluntary food intake when introduced into the blood stream, but that anandamide was ineffective: it neither decreased nor increased the food intake of the test animals.

US Patent Application 20050101542 describes the use of an antagonist of the CB1 receptor (a compound that has an activity at the CB1 receptor opposite to that of anandamide), together with fatty acid ethanolamides—but not anandamide—to reduce appetite. This clearly illustrates the concept of lowering the stimulation of the CB1 receptor as a method to reduce appetite.

On the other hand, it is known that CB1 receptor activation may lead to lower gastrointestinal motility, lower gastric acid secretion, and a delay in gastric emptying [Pertwee (2001)]. Again, stable alien ligands were often introduced orally, but the natural ligand anandamide only by injection.

### SUMMARY OF THE INVENTION

This invention relates to fatty acid ethanolamides for oral consumption. More specifically it relates to the oral consumption of the fatty acid ethanolamide anandamide (arachidonyl-ethanolamide or anandamide), and to mixtures that contain anandamide as a major component.

Surprisingly, it was found that oral consumption of anandamide led to a decreased appetite during ad libitum feeding, evidenced by a reduced intake of food for several hours. So in a preferred embodiment of the invention anandamide is used for oral intake preferably for reducing appetite or for giving a satiety effect.

According to one aspect of the invention anandamide is used for the manufacture of a nutraceutical or food for oral intake preferably for reducing appetite, for giving a satiety effect, for preventing or reducing inflammatory bowel disease or for preventing or reducing irritable bowel syndrome.

Thus anandamide is used for reducing appetite, for giving a satiety effect, for preventing or reducing inflammatory bowel disease or for preventing or reducing irritable bowel syndrome. According to another aspect of the invention a method is disclosed for reducing appetite, for giving a satiety effect, for preventing or reducing inflammatory bowel disease or for preventing or reducing irritable bowel syndrome which comprises the oral administering of anandamide to a subject in need of such treatment.

Furthermore the present invention relates to anandamide or a composition comprising anandamide for reducing appetite, for giving a satiety effect, for preventing or reducing inflammatory bowel disease or for preventing or reducing irritable bowel syndrome by oral intake. This composition preferably is a food or a beverage, a dietary supplement, a nutraceutical or a feed or pet food, including cereal bars, bakery items such as cakes and cookies, liquid foods such as soups or soup powders, beverages including non-alcoholic and alcoholic drinks as well as liquid preparations to be added to drinking water and liquid food, slimming foods, infant formula and clinical foods.

This composition comprises fatty acid ethanolamides of which anandamide preferably constitutes more than 20 wt %.

### DETAILED DESCRIPTION OF THE INVENTION

In the prior art, it is described that anandamide introduced by injection may increase food uptake. It is also known that anandamide may lower intestinal motility. Surprisingly, we have found that oral introduction of an endogenous ligand of the cannabinoid receptors led to a significant reduction of voluntary feed intake. We present the hypothesis that the supposedly short half-life of the endogenous compound makes it more suitable for local action in the digestive tract, in contrast to the more systemic effects of the stable exogenous cannabinoids, but this explanation is not to be taken as limiting for the invention. This hypothesis is presented to explain the present effect, however the present invention will not stand or fall with this hypothesis.

In the digestive tract, both anandamide and 2-arachidonyl glycerol are naturally present, as are a number of other fatty acid ethanolamides, such as oleoyl ethanolamide. CB1 receptors are present on myenteric and submucosal nerves of the enteric nervous system. Activation of these CB1 receptors inhibits gastrointestinal motility and gastric emptying, intestinal secretion and gastric acid secretion. We pose that the method of the present invention provides new strategies for the treatment of gut disorders, such as irritable bowel syndrome (IBS) and inflammatory bowel diseases (IBDs). In view of the presence of these ligands in the intestines, others have suggested that stable functional analogues would offer the best possibilities of providing an additional effect. Up to now, in the prior art, it is unclear whether oral administration of the more metabolically labile endogenous endocannabinoids could even produce such an effect.

Since we have shown that oral administration of an anandamide is effective in decreasing appetite, it follows that it is surprisingly possible to enhance local anandamide concentrations to a physiologically effective level. Hence, this also makes it possible to soothe the excessive gut motility in IBS, and to lower the inflammation associated with IBDs.

The present approach is different from all existing methods to reduce symptoms as anandamide will be effective within the gastrointestinal tract and will not (or minimally) become systemically available. This may minimize the occurrence of (serious) adverse events. Further, it will potentially be the first nature-identical substance that will be effective in IBS.

It is known that CB1 receptor activation may lead to lower gastrointestinal motility, lower gastric acid secretion, and a delay in gastric emptying [Pertwee (2001)]. Stable alien ligands were often introduced orally, but the natural ligand anandamide only by injection. A lowering of the gastrointestinal motility may be beneficial for relieving symptoms of Irritable Bowel Syndrome (IBS).

IBS is a collection of otherwise unexplained symptoms relating to a disturbance of the large bowel (colon). The symptoms include abdominal pain, cramps, bloating, diarrhoea, constipation and urgency to defecate. 3 subtypes of IBS have been identified: IBS-D (mainly diarrhoea), IBS-C (mainly constipation) and IBS-M (mixed—or alternating—constipation and diarrhoea). The American Gastroenterological Association has published a set of guidelines for tests which physicians should perform prior to diagnosing Irritable Bowel Syndrome. These tests are meant to exclude other causes, such as infection and colon cancer.

Estimated is that about 20 percent of the adult population in the US has symptoms of IBS, making it one of the most common disorders. It occurs more often in women than in men, and it begins before the age of 35 in about 50 percent of people.

Most people can control or minimize their symptoms with diet, stress management, and over-the-counter medication, like anti-diarrhoea medication or tablets containing fibers. However, for some people the symptoms are too severe or not reactive to life style changes. IBS can be disabling for them as they may be unable to work, attend social events, or even travel short distances. The few drugs that have been developed to treat IBS-D or IBS-C had to be withdrawn from the market shortly after introduction due to unforeseen serious side effects. This leaves the seriously affected people with no effective treatment.

Research into new drugs mainly focuses on serotonin. Serotonin is linked to normal gastrointestinal functioning. People with IBS are thought to have diminished receptor activity, causing abnormal levels of serotonin in the GI tract, which may lead to problems with bowel movement, motility and sensation (more sensitive pain receptors in GI tract). Drugs in development either look to block serotonin to treat IBS-D, or to enhance the effect of serotonin to treat constipation.

It has also been suggested that pharmacological modulation of the cannabinoid system may provide new therapeutics for the treatment of a number of gastrointestinal diseases, including IBS [Di Carlo & Izzo (2003) Cannabinoids for Gastrointestinal Diseases; Potential Therapeutic Applications; Expert Opin. Investig. Drugs 12:39]. However, the use of natural endocannabinoids introduced via an oral route has not been considered for this purpose.

Similarly, it is known that an inflammatory condition of the gut is associated with increased endocannabinoid levels [d'Argenio et al. (2006) Up-regulation of Anandamide Levels as an Endogenous Mechanism and a Pharmacological Strategy to Limit Colon Inflammation; FASEB J. 20:1279-1294].

Inflammatory bowel disease includes a number of chronic, relapsing inflammatory disorders involving the gastrointestinal tract, of which the most known are ulcerative colitis and Crohn's disease.

Ulcerative colitis is an inflammatory disease of the colon, in which the inner lining (mucosa) of the intestine becomes inflamed and develops ulcers. The disease is often the most severe in the rectal area, which can cause frequent diarrhea. Mucus and blood generally appear in the stool if the lining of the colon is damaged.

Crohn's disease differs from ulcerative colitis in the areas of the bowel it involves—it most commonly affects the last part of the small intestine (the terminal ileum) and parts of the colon. However, Crohn's disease is not limited to these areas and can attack any part of the digestive tract from mouth to anus. Crohn's disease causes inflammation that extends much deeper into the layers of the intestinal wall than ulcerative colitis does; Crohn's disease generally tends to involve the entire bowel wall, whereas ulcerative colitis affects only the lining of the bowel.

It is estimated that more than 600,000 people in the United States have some form of inflammatory bowel disease.

The pathogenesis of inflammatory bowel disease is not known. A genetic predisposition has been suggested, and a host of environmental factors, including bacterial, viral and, perhaps, dietary antigens, can trigger an ongoing enteric inflammatory cascade. As mentioned previously, inflammatory conditions have been associated with increased anandamide levels. Whether the elevated concentrations of anandamide are seen as a consequence of the inflammation (e.g. by cell death or leakage from cells) or that inflammation stimulated cells to produce and release more anandamide is unknown. It may also be possible that patients with inflammatory bowl disease are less sensitive to anandamide or that binding of anandamide to the CB1 and CB2 receptor is decreased. It has been found that the condition of animals with experimentally induced inflammation in the gut could be improved by treatment with substances that are effective in increasing the level of anandamide [d'Argenio et al. (2006)]. However, it is not known whether this is a causal effect, whether oral administration of anandamide would be equally effective, and whether this would also be an effective therapy in animals or humans with spontaneously evolved inflammatory diseases of the gut.

Anandamide is a substance that is naturally produced by the body, amongst others in the gut. Its function in the gut is not completely understood, but may include influencing gastrointestinal motility.

Anandamide taken orally as part of a food/drink or as a supplement or drug may be helpful in the treatment of IBS (specifically the subtype IBS-D—people who suffer from diarrhoea) and/or may be used as prophylactic in patients with IBS-D. In view of the chronic nature of the disease, prophylactic use via food and beverages, dietary supplement or nutraceutical may be help to reduce the number of episodes. We hypothesize that a mild reduction in GI motility may ‘balance’ the intestines better and may have a positive effect on the number of episodes.

Good sources of anandamide—or of other fatty acid ethanolamides, fatty acid amides or fatty acid 2-monoglycerides—have not been found in nature. Early reports mention its presence in—for instance—chocolate, but the levels were extremely low, and subsequent work has not taken these sources into account. Hence, for commercial application such molecules must be synthesized, either chemically, enzymatically, by biotransformation, or by fermentation.

Fatty acid ethanolamides may be considered as being composed of two parts: an ethanolamine moiety, N-substituted with a second moiety, the fatty acid. Both parts may be used as raw material in an essentially pure form, but they may also be comprised in a more complex molecule. When only using pure compounds, one would couple the free fatty acid to free ethanolamine. More complex compounds that could be used are acyl-glycerol lipids, phospholipids, sphingolipids, glycolipids, and other sources containing fatty acid residues. In general, these compounds are esters of fatty acids. With respect to the ethanolamine moiety, it may be considered that these are comprised in many polar lipids, such phospholipids, again in esterified form. One may even envisage the use of fatty acid ethanolamides themselves, to achieve a trans-esterification using other ethanolamides, fatty acids, ethanolamine, or the esters of fatty acids and ethanolamine. In a particularly useful method, lipids isolated from biological sources could be used as raw materials.

Anandamide is the compound central to this invention, on account of its satiety-inducing effect. However, it can also be seen as a representative molecule for classes of compounds that could contribute to a similar effect.

Anandamide is a representative of the fatty acid ethanolamides. Other fatty acid ethanolamides, even those that have been shown not to be ligands of the CB1 and CB2 receptors, may exert a positive effect on the action of anandamide. Because of their similar molecular structure, fatty acid ethanolamides inhibit each others breakdown by the fatty acid amide hydrolase. Therefore, the presence of other fatty acid ethanolamides has a protective effect on anandamide, thereby increasing its potency. Anandamide is also a representative of the CB-receptor ligands. Many synthetic ligands have a different pharmacokinetic profile, and have therefore not the same effect on the physiology. However, other natural ligands, the most noteworthy of which is 2-arachidonyl-monoglyceride, have similar properties, being also metabolically labile, thereby exerting a transient effect on the receptor. Therefore, the use of metabolically labile CB-receptor ligands falls within the scope of this invention.

Anandamide may be applied alone, which is to say: in the absence of other ethanolamides, and in the absence of other compounds that are CB receptor ligands. However, anandamide may also be applied as part of a mixture of fatty acid ethanolamides. Preferably, the anandamide is the most abundant fatty acid ethanolamide in the mixture. More preferably, the anandamide comprises more than 20 wt % of the mixture, more preferably more than 40 wt %, still more preferably more than 50 wt %, even still more preferably more than 60 wt %, most preferably more than 80 wt %.

Anandamide may also be applied as part of a mixture of CB receptor ligands. Preferably, the anandamide is the most abundant fatty acid CB receptor ligand in the mixture. More preferably, the anandamide comprises more than 25 wt % of the mixture, more preferably more than 50 wt %, most preferably more than 75 wt %. The anandamide-containing composition may also be combined with other substances that have a satiety effect, but that are not fatty acid ethanolamides or CB receptor ligands, such as bioactive peptides, protein hydrolysates, certain lipids, etc.

It is a defining feature of the present invention that the anandamide-containing preparation is introduced into the body via the gastro-intestinal tract. In a clinical or experimental setting this may be done by direct introduction into the stomach or intestines, in a food-related setting the introduction will generally be through the mouth. In principle, anandamide-containing preparation can be used in all organisms that have a CB receptor system. More preferably the organism is a mammal, even more preferably a human.

The term dietary supplement as used herein denotes a product taken by mouth that contains a compound or mixture of compounds intended to supplement the diet. The compound or mixture of compounds in these products may include: vitamins, minerals, herbs or other botanicals and amino acids. Dietary supplements can also be extracts or concentrates, and may be found in many forms such as tablets, capsules, soft gels, gel caps, liquids, or powders.

The term nutraceutical as used herein denotes the usefulness in both the nutritional and pharmaceutical field of application. The nutraceutical compositions according to the present invention may be in any form that is suitable for administrating to the animal body including the human body, especially in any form that is conventional for oral administration, e.g. in solid form such as (additives/supplements for) food or feed, food or feed premix, tablets, pills, granules, dragées, capsules, and effervescent formulations such as powders and tablets, or in liquid form such as solutions, emulsions or suspensions as e.g. beverages, pastes and oily suspensions. Controled (delayed) release formulations incorporating anandamide according to the invention also form part of the invention. Furthermore, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns. The nutraceutical can further comprise usual additives, for example sweeteners, flavors, sugar, fat, emulgators, preservatives. The nutrition can also comprise other active components, such as (hydrolysed) proteins as described for example in WO02/45524. Also anti-oxidants can be present in the nutrition, for example flavonoids, carotenoids, ubiquinones, rutin, lipoic acid, catalase, glutatione (GSH) and vitamins, such as for example C and E or their precursors.

Anandamide is advantageously present in an effective amount. Generally between 1 mg and 3 gram of the compound is effective per serving to assort an effect. Preferably more than 10 mg, even more preferably more than 100 mg, and most preferably more than 250 mg per serving is used. Preferably less than 2.5 gram, even more preferably less than 1 gram, and most preferably less than 750 mg per serving is used. This depends on a number of factors, such as weight, age, dietary habits and exercise intensity.

Preferably 1 or 2 servings are used each day. Therefore, preferably 100 mg to 2 gram per day of anandamide are orally taken by a person to obtain the effects of the present invention.

The nutraceutical comprising anandamide can be consumed before, during or after the meal.

In addition to nutraceutical products suitable for consumption of humans, it is also possible to use anandamide in feed for animals including pet food.

There are different methods, in which the consumption of the anandamide-containing preparation can be adjusted to the dietary pattern. Because it takes a certain time before the anandamide will be effective in reducing the appetite, consumption of anandamide just before or during a meal will not have an effect on the consumption of that meal. Rather, it will reduce the appetite some time after that meal and/or during the subsequent meal. It follows, that the anandamide-containing preparation may also be consumed between meals or some time before a meal.

The invention is hereafter elucidated with the following non-limiting examples.

### LEGENDS TO THE FIGURES

FIG. 1. Cumulative food intake of rats after anandamide infusion at t=0.

### EXAMPLES

### Example 1

An experiment was conducted to measure the effect of different levels of anandamide intake on food intake in rats. Thirteen adult male Sprague-Dawley rats (Charles River, Germany) were surgically equipped with a chronic gastric cannula. Animals were individually housed in plastic cages (42×22×36 cm) with stainless steel grid floors in a temperature-controlled (22±2° C.) room. The 12:12-h light-dark cycle was inversed (lights off 09:00 h).

Rats had free access to water and restricted access to ground extruded feed (No. 3436, Provimi Kliba-nafag, Kaiseraugst, Switzerland). To prevent spoilage of food, it was placed in cups at the end of a tunnel (5×10×30 cm) that protruded from the side of the cage, with its bottom 5 cm above the cage floor. Food cups were mounted on electronic balances (Mettler PM 3000, Switzerland) that were interfaced with a computer (Olivetti M 300, Germany). Weights of the food cups were recorded the every 30 s, enabling continuous measurement of spontaneous feeding patterns. Anandamide (98% purity, dissolved in ethanol) was obtained from Cayman Chemical Company, Michigan, USA). It was stored deep-frozen, until the experiment. Than the proper quantity was mixed into sunflower oil.

Before starting the experimental period, rats (394±7 g) were adapted to the restricted food access. One day prior to the test, they were also adapted to infusion of sunflower oil. The experimental period lasted five consecutive days. During the experiment, rats were deprived from food each day for 15 h (18.00-9.00 h). At 09.00, they were intragastrically infused with 0.5 ml sunflower oil containing 0, 5, or 15 mg Anandamide/mL, after which the cannulas were flushed with 1.5 mL saline. Each of these doses was given to three rats. One rat was excluded from the experiment after 2 days, and a spare animal was included.

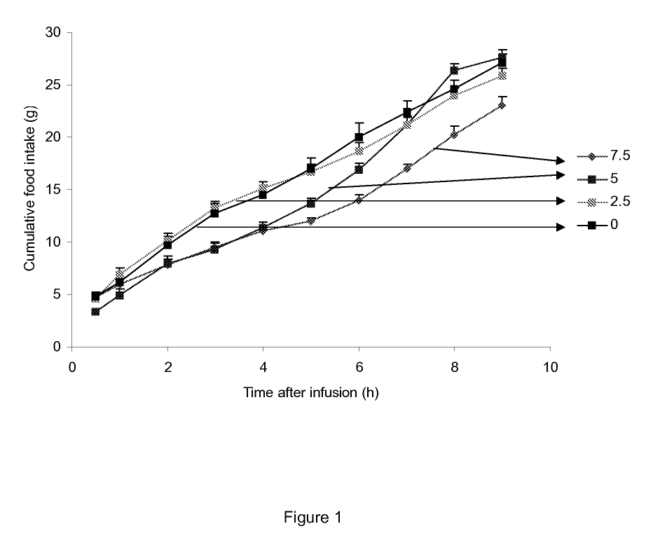

In FIG. 1 the mean cumulative food intake for the four treatments is presented. The 9-h cumulative food intake was 27.1, 25.8, 27.6, and 23.0 g for addition of 0, 2.5, 5.0, and 7.5 mg anandamide/d, respectively. The 7.5 mg treatment was significantly different from the other treatments.

Calculated as a mean over the 9-h period, food intake was significantly lower with 5.0 and 7.5 mg, than with 0 or 2.5 mg anandamide. The lowest dose (2.5 mg/d) did not affect food intake compared to the control. Compared to the control, 5.0 mg anandamide reduced food intake significantly (P<0.001) after 3 through 6 hours after infusion, but the difference disappeared during the later stage of the experiment. The lower food intake persisted until 9 h post-infusion, however, at the highest anandamide dose (7.5 mg/d).

Conclusion: Anandamide significantly reduced food intake in rats after intragastric infusion of 5.0 or 7.5 mg. With 5.0 mg, the effect was significant from 3 through 6 h post infusion, with 7.5 mg is remained significant to 9 h post infusion. Anandamide can, thus, reduce food intake.

1. The use of anandamide for the manufacture of a nutraceutical for oral intake preferably for reducing appetite, for giving a satiety effect, for preventing or reducing inflammatory bowel disease or for preventing or reducing irritable bowel syndrome.

2. The use of anandamide for reducing appetite, for giving a satiety effect, for preventing or reducing inflammatory bowel disease or for preventing or reducing irritable bowel syndrome.

3. The use of claim 1 which comprises the use of 0.1 mg to 3 gram of anandamide per serving.

4. The use of claim 1 which comprises the oral intake of 100 mg to 2 gram per day of anandamide.

5. A method of treatment for reducing appetite, for giving a satiety effect, for preventing or reducing inflammatory bowel disease or for preventing or reducing irritable bowel syndrome which comprises the oral administering anandamide to a subject in need of such treatment.

6. Anandamide for oral intake for reducing appetite, for giving a satiety effect, for preventing or reducing inflammatory bowel disease or for preventing or reducing irritable bowel syndrome.

7. A composition for oral intake comprising anandamide for reducing appetite, for giving a satiety effect, for preventing or reducing inflammatory bowel disease or for preventing or reducing irritable bowel syndrome.

8. A composition of claim 7 which comprises the use of 0.1 mg to 3 gram of anandamide.

9. A composition of claim 7 which is a food or a beverage, as a dietary supplement, as a nutraceutical or in feed or pet food, including cereal bars, bakery items such as cakes and cookies, liquid foods such as soups or soup powders, beverages including non-alcoholic and alcoholic drinks as well as liquid preparations to be added to drinking water and liquid food, slimming foods, infant formula and clinical foods.

10. A composition of comprising fatty acid ethanolamides of which anandamide constitutes more than 20 wt %, and preferably more than 80 Wt %.
